(19) United States
(12) Patent Application Publication
(10) Pub. No.: US 2024/0131182 A1
(43) Pub. Date: Apr. 25, 2024

(54) GOLD CLUSTERS, COMPOSITIONS, AND METHODS FOR TREATMENT OF DEPRESSION

(71) Applicant: SHENZHEN PROFOUND VIEW PHARMACEUTICAL TECHNOLOGY CO., LTD., SHENZHEN (CN)

(72) Inventor: Taolei SUN, WUHAN (CN)

(73) Assignee: SHENZHEN PROFOUND VIEW PHARMACEUTICAL TECHNOLOGY CO., LTD., SHENZHEN, GD (CN)

(21) Appl. No.: 18/546,269

(22) PCT Filed: Apr. 24, 2021
(86) PCT No.: PCT/CN2021/089648

§ 371 Date: Aug. 11, 2023

(51) Int. Cl. A61K 47/69; A61K 33/242; A61K 47/54; A61K 47/64; A61P 25/24

(52) U.S. Cl. CPC A61K 47/6929; A61K 33/242; A61K 47/542; A61K 47/64; A61P 25/24

(57) ABSTRACT

Ligand-bound gold clusters and compositions comprising the ligand-bound gold clusters are used for treating depression and manufacturing a medicament for treatment of depression. Methods for treating depression.

### FIELD OF THE INVENTION

The present invention relates to the technical field of mental illness, particularly to ligand-bound gold clusters (AuCs), composition comprising the ligand-bound AuCs, use of the ligand-bound AuCs to prepare medications for treatment of depression, and methods employing the ligand-bound AuCs and composition for treatment of depression.

### BACKGROUND OF THE INVENTION

Depression is a mental illness with a high prevalence in humans reaching 21% of the worldwide population, and causes severe symptoms including sadness, anger, frustration, hopelessness, anxiety, irritability, lack of motivation, and feelings of guilt.

While a variety of factors have been suspected to be involved including biological differences, brain chemistry, hormones, inherited traits, and chronic inflammation, the exact factor that causes depression or the exact mechanism by which any factor causes depression is not known, imposing a grave challenge to research and development of treatment for depression.

The currently available medicines that treat depression are antidepressants that directly affect the chemistry of the brain and presumably achieve their therapeutic effects by correcting the chemical dysregulation that is causing the depression. The antidepressants include tricyclic antidepressants, selective serotonin reuptake inhibitors (SSRIs; e.g., fluoxetine, paroxetine, sertraline, fluvoxamine, citalopram and escitalopram), and serotonin and norepinephrine reuptake inhibitors (SNRI, representatives including venlafaxine and duloxetine).

Studies have attempted to investigate the effects of nanoparticles on depression. Nanosized zinc oxide (NanoZnO) with a size of 20-80 nm decreased the immobility time of forced swim test (FST) in a lipopolysaccharides (LPS)-induced depression mouse model (Xie 2012). Iron nanoparticles (INP) with a size of 20 nm improved depression symptoms in an LPS-induced depression rat model (Saeidienik 2018). However, Nano silver (nanoAg) with a size of 10 nm by oral administration at a dose of 0.2 mg/kg body weight induced morphological disturbances in myelin sheaths, showing toxicity to central nerve system (CNS) in rat (Dabrowska-Bouta 2016). Al₂O₃ nanoparticles (NPs) through a respiratory route caused depression-like behavior in female mice (Zhang 2015). It is evident that prior studies do not provide any consensus or guidance on the effects of nanoparticles on depression.

There remains a need for effective method and medications for treatment of depression.

### SUMMARY OF THE INVENTION

The present invention provides ligand-bound gold clusters for use to treat the depression in a subject, the method of treating the depression in a subject with ligand-bound gold clusters, and the use of ligand-bound gold clusters for manufacture of medicament for treatment of the depression in a subject.

Certain embodiments of the present invention use of a ligand-bound gold cluster to treat the depression in a subject, wherein the ligand-bound gold cluster comprises a gold core; and a ligand bound to the gold core.

In certain embodiments of the treatment use, the gold core has a diameter in the range of 0.5-3 nm. In certain embodiments, the gold core has a diameter in the range of 0.5-2.6 nm.

In certain embodiments of the treatment use, the ligand is one selected from the group consisting of L-cysteine and its derivatives, D-cysteine and its derivatives, cysteine-containing oligopeptides and their derivatives, and other thiol-containing compounds.

In certain embodiments of the treatment use, the L-cysteine and its derivatives are selected from the group consisting of L-cysteine, N-isobutyryl-L-cysteine (L-NIBC), and N-acetyl-L-cysteine (L-NAC), and the D-cysteine and its derivatives are selected from the group consisting of D-cysteine, N-isobutyryl-D-cysteine (D-NIBC), and N-acetyl-D-cysteine (D-NAC).

In certain embodiments of the treatment use, the cysteine-containing oligopeptides and their derivatives are cysteine-containing dipeptides, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

In certain embodiments of the treatment use, the cysteine-containing oligopeptides and their derivatives are cysteine-containing tripeptides, wherein the cysteine-containing tripeptides are selected from the group consisting of glycine-L(D)-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

In certain embodiments of the treatment use, the cysteine-containing oligopeptides and their derivatives are cysteine-containing tetrapeptides, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GC SR).

In certain embodiments of the treatment use, the cysteine-containing oligopeptides and their derivatives are cysteine-containing pentapeptide, wherein the cysteine-containing pentapeptides are selected from the group consisting of Cysteine-Aspartic acid-Glutamic acid-Valine-Aspartic acid (CDEVD) and Aspartic acid-Glutamic acid-Valine-Aspartic acid-Cysteine (DEVDC).

In certain embodiments of the treatment use, the other thiol-containing compounds are selected from the group consisting of 1-[(2S)-2-methyl-3-thiol-1-oxopropyl]-L(D)-proline, thioglycollic acid, mercaptoethanol, thiophenol, D-3-trolovol, N-(2-mercaptopropionyl)-glycine, dodecyl mercaptan, 2-aminoethanethiol (CSH), 3-mercaptopropionic acid (MPA), and 4-mercaptobenzoic acid (p-MBA).

Certain embodiments of the present invention use a ligand-bound gold cluster for manufacture of a medicament for the treatment of the depression in a subject, wherein ligand-bound gold cluster comprises a gold core; and a ligand bound the gold core.

In certain embodiments of the manufacture use, the gold core has a diameter in the range of 0.5-3 nm. In certain embodiments, the gold core has a diameter in the range of 0.5-2.6 nm.

In certain embodiments of the manufacture use, the ligand is one selected from the group consisting of L-cysteine and its derivatives, D-cysteine and its derivatives, cysteine-containing oligopeptides and their derivatives, and other thiol-containing compounds.

In certain embodiments of the manufacture use, the L-cysteine and its derivatives are selected from the group consisting of L-cysteine, N-isobutyryl-L-cysteine (L-NIBC), and N-acetyl-L-cysteine (L-NAC), and the D-cysteine and its derivatives are selected from the group consisting of D-cysteine, N-isobutyryl-D-cysteine (D-NIBC), and N-acetyl-D-cysteine (D-NAC).

In certain embodiments of the manufacture use, the cysteine-containing oligopeptides and their derivatives are cysteine-containing dipeptides, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

In certain embodiments of the manufacture use, the cysteine-containing oligopeptides and their derivatives are cysteine-containing tripeptides, wherein the cysteine-containing tripeptides are selected from the group consisting of glycine-L(D)-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

In certain embodiments of the manufacture use, the cysteine-containing oligopeptides and their derivatives are cysteine-containing tetrapeptides, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GC SR).

In certain embodiments of the manufacture use, the cysteine-containing oligopeptides and their derivatives are cysteine-containing pentapeptide, wherein the cysteine-containing pentapeptides are selected from the group consisting of Cysteine-Aspartic acid-Glutamic acid-Valine-Aspartic acid (CDEVD) and Aspartic acid-Glutamic acid-Valine-Aspartic acid-Cysteine (DEVDC).

In certain embodiments of the manufacture use, the other thiol-containing compounds are selected from the group consisting of 1-[(2S)-2-methyl-3-thiol-1-oxopropyl]-L(D)-proline, thioglycollic acid, mercaptoethanol, thiophenol, D-3-trolovol, N-(2-mercaptopropionyl)-glycine, dodecyl mercaptan, 2-aminoethanethiol (CSH), 3-mercaptopropionic acid (MPA), and 4-mercaptobenzoic acid (p-MBA).

The objectives and advantages of the invention will become apparent from the following detailed description of preferred embodiments thereof in connection with the accompanying drawings.

### DESCRIPTION OF THE DRAWINGS

Preferred embodiments according to the present invention will now be described with reference to the Figures, in which like reference numerals denote like elements.

FIG. 1 shows ultraviolet-visible (UV) spectrums, transmission electron microscope (TEM) images and particle size distribution diagrams of ligand L-NIBC-modified gold nanoparticles (L-NIBC-AuNPs) with different particle sizes.

FIG. 2 shows ultraviolet-visible (UV) spectrums, TEM images and particle size distribution diagrams of ligand L-NIBC-bound gold clusters (L-NIBC-AuCs) with different particle sizes.

FIG. 3 shows infrared spectra of L-NIBC-AuCs with different particle sizes.

FIG. 4 shows UV, infrared, TEM, and particle size distribution diagrams of ligand CR-bound gold clusters (CR-AuCs).

FIG. 5 shows UV, infrared, TEM, and particle size distribution diagrams of ligand RC-bound gold clusters (RC-AuCs).

FIG. 6 shows UV, infrared, TEM, and particle size distribution diagrams of ligand 1-[(2S)-2-methyl-3-thiol-1-oxopropyl]-L-proline (i.e., Cap)-bound gold clusters (Cap-AuCs).

FIG. 7 shows UV, infrared, TEM, and particle size distribution diagrams of ligand GSH-bound gold clusters (GSH-AuCs).

FIG. 8 shows UV, infrared, TEM, and particle size distribution diagrams of ligand D-NIBC-bound gold clusters (D-NIBC-AuCs).

FIG. 9 shows UV, infrared, TEM, and particle size distribution diagrams of ligand L-cysteine-bound gold clusters (L-Cys-AuCs).

FIG. 10 shows UV, infrared, TEM, and particle size distribution diagrams of ligand 2-aminoethanethiol-bound gold clusters (CSH-AuCs).

FIG. 11 shows UV, infrared, TEM, and particle size distribution diagrams of ligand 3-mercaptopropionic acid-bound gold clusters (MPA-AuCs).

FIG. 12 shows UV, infrared, TEM, and particle size distribution diagrams of ligand 4-mercaptobenoic acid-bound gold clusters (p-MBA-AuCs).

FIG. 13 shows UV, TEM, and particle size distribution diagrams of ligand 4-Cysteine-Aspartic acid-Glutamic acid-Valine-Aspartic acid (CDEVD)-bound gold clusters (CDEVD-AuCs).

FIG. 14 shows UV, TEM, and particle size distribution diagrams of ligand 4-Aspartic acid-Glutamic acid-Valine-Aspartic acid-Cysteine (DEVDC)-bound gold clusters (DEVDC-AuCs).

FIG. 15 is a bar graph showing the results of forced swimming tests.

FIG. 16 is a bar graph showing the results of tail suspension tests.

FIG. 17 shows the results of social behavior test of mice with chronic social stress: A, the social interaction time T1 of normal mice and model mice in the social interaction zone in the first stage test; B, the social interaction time T2 of normal mice and model mice in social interaction zone in the second stage test; C, the social interaction ratio T2/T1 of normal mice and model mice in social behavior test. The data were shown in Mean±SEM, ** P<0.01 and *** P<0.001, compared with normal mice in the normal control group.

FIG. 18 shows the results of social behavior test of mice in A1 drug administration group: A, in the first stage test, the social interaction time T1 of the normal control group, model control group and A1 drug administration group mice in the social interaction zone; B, in the second stage, the social interaction time T2 of the normal control group, model control group and A1 drug administration group mice in the social interaction zone; C, the social interaction ratio T2/T1 of the normal control group, model control group and A1 drug administration group mice in social behavior test. The data were shown in Mean±SEM, #P<0.05, compared with the normal control group, * P<0.05, compared with the model control group.

FIG. 19 shows the results of elevated cross maze test of mice in A4 drug administration group: A, the movement time of the normal control group, model control group and A4 drug administration group mice in the open arm; B. the movement distance of the normal control group, model control group and A4 drug administration group mice in the open arm; C, the shuttling times of the normal control group, model control group and A4 drug administration group mice in the open arm; D, the percentage of open arm time to open arm+closed arm time (TO %) of the normal control group, model control group and A4 drug administration group mice; E, the percentage of open arm movement distance to total movement distance (DO %) of the normal control group, model control group and A4 drug administration group mice; F, the percentage of open arm shuttling times and total shuttling times (EO %) of the normal control group, model control group and A4 drug administration group mice. The data in the figure are represented by Mean±SEM, #P<0.05, compared with the normal control group, * P<0.05, compared with the model control group.

### DETAILED DESCRIPTION OF THE EMBODIMENTS

The present invention may be understood more readily by reference to the following detailed description of certain embodiments of the invention.

Throughout this application, where publications are referenced, the disclosures of these publications are hereby incorporated by reference, in their entireties, into this application in order to more fully describe the state of art to which this invention pertains.

As used herein, “administering” means oral (“po”) administration, administration as a suppository, topical contact, intravenous (“iv”), intraperitoneal (“ip”), intramuscular (“im”), intralesional, intrahippocampal, intracerebroventricular, intranasal or subcutaneous (“sc”) administration, or the implantation of a slow-release device e.g., a mini-osmotic pump or erodible implant, to a subject. Administration is by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (i.e. other than through the alimentary tract, such as intramuscular, intravenous, intra-arterial, transdermal and subcutaneous) administration, with the proviso that, as used herein, systemic administration does not include direct administration to the brain region by means other than via the circulatory system, such as intrathecal injection and intracranial administration.

As used herein, the terms “treating” and “treatment” refer to delaying the onset of, retarding or reversing the progress of, or alleviating or preventing either the disease or condition to which the term applies, or one or more symptoms of such disease or condition.

The terms “patient,” “subject” or “individual” interchangeably refers to a mammal, for example, a human or a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster, guinea pig).

Gold clusters (AuCs) are a special form of gold existing between gold atoms and gold nanoparticles. AuCs have a size smaller than 3 nm, and are composed of only several to a few hundreds of gold atoms, leading to the collapse of face-centered cubic stacking structure of gold nanoparticles. As a result, AuCs exhibit molecule-like discrete electronic structures with distinct HOMO-LUMO gap unlike the continuous or quasi-continuous energy levels of gold nanoparticles. This leads to the disappearance of surface plasmon resonance effect and the corresponding plasmon resonance absorption band (520±20 nm) at UV-Vis spectrum that possessed by conventional gold nanoparticles.

The present invention provides a ligand-bound AuC.

In certain embodiments, the ligand-bound AuC comprises a ligand and a gold core, wherein the ligand is bound to the gold core. The binding of ligands with gold cores means that ligands form stable-in-solution complexes with gold cores through covalent bond, hydrogen bond, electrostatic force, hydrophobic force, van der Waals force, etc. In certain embodiments, the diameter of the gold core is in the range of 0.5-3 nm. In certain embodiments, the diameter of the gold core is in the range of 0.5-2.6 nm.

In certain embodiments, the ligand of the ligand-bound AuC is a thiol-containing compound or oligopeptide. In certain embodiments, the ligand bonds to the gold core to form a ligand-bonded AuC via Au—S bond.

In certain embodiments, the ligand is, but not limited to, L-cysteine, D-cysteine, or a cysteine derivative. In certain embodiments, the cysteine derivative is N-isobutyryl-L-cysteine (L-NIBC), N-isobutyryl-D-cysteine (D-NIBC), N-acetyl-L-cysteine (L-NAC), or N-acetyl-D-cysteine (D-NAC).

In certain embodiments, the ligand is, but not limited to, a cysteine-containing oligopeptide and its derivatives. In certain embodiments, the cysteine-containing oligopeptide is a cysteine-containing dipeptide. In certain embodiments, the cysteine-containing dipeptide is L(D)-cysteine-L(D)-arginine dipeptide (CR), L(D)-arginine-L(D)-cysteine dipeptide (RC), or L(D)-cysteine-L-histidine dipeptide (CH). In certain embodiments, the cysteine-containing oligopeptide is a cysteine-containing tripeptide. In certain embodiments, the cysteine-containing tripeptide is glycine-L(D)-cysteine-L(D)-arginine tripeptide (GCR), L(D)-proline-L(D)-cysteine-L(D)-arginine tripeptide (PCR), or L(D)-glutathione (GSH). In certain embodiments, the cysteine-containing oligopeptide is a cysteine-containing tetrapeptide. In certain embodiments, the cysteine-containing tetrapeptide is glycine-L(D)-serine-L(D)-cysteine-L(D)-arginine tetrapeptide (GSCR) or glycine-L(D)-cysteine-L(D)-serine-L(D)-arginine tetrapeptide (GC SR). In certain embodiments, the cysteine-containing oligopeptide is a cysteine-containing pentapeptide. In certain embodiments, the cysteine-containing pentapeptide is Cysteine-Aspartic acid-Glutamic acid-Valine-Aspartic acid (CDEVD), or Aspartic acid-Glutamic acid-Valine-Aspartic acid-Cysteine (DEVDC).

In certain embodiments, the ligand is a thiol-containing compound. In certain embodiments, thiol-containing compound is 1-[(2S)-2-methyl-3-thiol-1-oxopropyl]-L(D)-proline, thioglycollic acid, mercaptoethanol, thiophenol, D-3-trolovol, dodecyl mercaptan, 2-aminoethanethiol (CSH), 3-mercaptopropionic acid (MPA), or 4-mercaptobenzoic acid (p-MBA).

The present invention provides a pharmaceutical composition for the treatment of depression. In certain embodiments, the subject is human. In certain embodiments, the subject is a pet animal such as a dog.

In certain embodiments, the pharmaceutical composition comprises a ligand-bound AuC as disclosed above and a pharmaceutically acceptable excipient. In certain embodiments, the excipient is phosphate-buffered solution, or physiological saline.

The present invention provides a use of the above disclosed ligand-bound AuCs for manufacturing a medication for the treatment of depression.

The present invention provides a use of the above disclosed ligand-bound AuCs for treating a subject with depression, or a method for treating a subject with depression using the above disclosed ligand-bound AuCs. In certain embodiments, the method for treatment comprises administering a pharmaceutically effective amount of ligand-bound AuCs to the subject. The pharmaceutically effective amount can be ascertained by routine in vivo studies. In certain embodiments, the pharmaceutically effective amount of ligand-bound AuCs is a dosage of at least 0.001 mg/kg/day, 0.005 mg/kg/day, 0.01 mg/kg/day, 0.05 mg/kg/day, 0.1 mg/kg/day, 0.5 mg/kg/day, 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 15 mg/kg/day, 20 mg/kg/day, 30 mg/kg/day, 40 mg/kg/day, 50 mg/kg/day, 60 mg/kg/day, 70 mg/kg/day, 80 mg/kg/day, 100 mg/kg/day, 200 mg/kg/day, 300 mg/kg/day, 400 mg/kg/day, 500 mg/kg/day, 600 mg/kg/day, 700 mg/kg/day, 800 mg/kg/day, 900 mg/kg/day, or 1000 mg/kg/day.

The following examples are provided for the sole purpose of illustrating the principles of the present invention; they are by no means intended to limit the scope of the present invention.

### EMBODIMENTS

### Embodiment 1. Preparation of Ligand-Bound AuCs

1.1 Dissolving HAuCl₄ in methanol, water, ethanol, n-propanol, or ethyl acetate to get a solution A in which the concentration of HAuCl₄ is 0.01˜0.03M;
1.2 Dissolving a ligand in a solvent to get a solution B in which the concentration of the ligand is 0.01˜0.18M; the ligand includes, but not limited to, L-cysteine, D-cysteine and other cysteine derivatives such as N-isobutyryl-L-cysteine (L-NIBC), N-isobutyryl-D-cysteine (D-NIBC), N-acetyl-L-cysteine (L-NAC), and N-acetyl-D-cysteine (D-NAC), cysteine-containing oligopeptides and their derivatives including, but not limited to, dipeptides, tripeptide, tetrapeptide, pentapeptide, and other peptides containing cysteine, such as L(D)-cysteine-L(D)-arginine dipeptide (CR), L(D)-arginine-L(D)-cysteine dipeptide (RC), L(D)-cysteine L(D)-histidine (CH), glycine-L(D)-cysteine-L(D)-arginine tripeptide (GCR), L(D)-proline-L(D)-cysteine-L(D)-arginine tripeptide (PCR), L(D)-glutathione (GSH), glycine-L(D)-serine-L(D)-cysteine-L(D)-arginine tetrapeptide (GSCR), glycine-L(D)-cysteine-L(D)-serine-L(D)-arginine tetrapeptide (GCSR), Cysteine-Aspartic acid-Glutamic acid-Valine-Aspartic acid pentapeptide (CDEVD) and Aspartic acid-Glutamic acid-Valine-Aspartic acid-Cysteine pentapeptide (DEVDC), and other thiol-containing compounds, such as one or more of 1-[(2S)-2-methyl-3-thiol-1-oxopropyl]-L(D)-proline, thioglycollic acid, mercaptoethanol, thiophenol, D-3-trolovol, dodecyl mercaptan, 2-aminoethanethiol (CSH), 3-mercaptopropionic acid (MPA), and 4-mercaptobenzoic acid (p-MBA); the solvent is one or more of methanol, ethyl acetate, water, ethanol, n-propanol, pentane, formic acid, acetic acid, diethyl ether, acetone, anisole, 1-propanol, 2-propanol, 1-butanol, 2-butanol, pentanol, butyl acetate, tributyl methyl ether, isopropyl acetate, dimethyl sulfoxide, ethyl formate, isobutyl acetate, methyl acetate, 2-methyl-1-propanol and propyl acetate;
1.3 Mixing solution A and solution B so that the mole ratio between HAuCl₄ and ligand is 1:(0.01˜100), stirring them in an ice bath for 0.1˜48 h, adding 0.025˜0.8M NaBH 4 water, ethanol or methanol solution, continuing to stir in an ice water bath and react for 0.1˜12 h. The mole ratio between NaBH₄ and ligand is 1:(0.01˜100);
1.4 Using MWCO 3K˜30K ultrafiltration tubes to centrifuge the reaction solution at 8000˜17500 r/min by gradient for 10˜100 min after the reaction ends to obtain ligand-bound AuCs precipitate in different average particle sizes. The aperture of the filtration membranes for ultrafiltration tubes of different MWCOs directly decides the size of ligand-bound AuCs that can pass the membranes. This step may be optionally omitted;
1.5 Dissolving the ligand-bound AuCs precipitate in different average particle sizes obtained in step (1.4) in water, putting it in a dialysis bag and dialyzing it in water at room temperature for 1˜7 days;
1.6 Freeze-drying ligand-bound AuCs for 12˜24 h after dialysis to obtain a powdery or flocculant substance, i.e., ligand-bound AuCs.

As detected, the particle size of the powdery or flocculant substance obtained by the foregoing method is smaller than 3 nm (distributed in 0.5-2.6 nm in general). No obvious absorption peak at 520 nm. It is determined that the obtained powder or floc is ligand-bound AuCs.

### Embodiment 2. Preparation and Characterization of AuCs Bound with Different Ligands

2.1 Preparation of L-NIBC-Bound AuCs, i.e. L-NIBC-AuCs

Taking ligand L-NIBC for example, the preparation and confirmation of AuCs bound with ligand L-NIBC are detailed.
2.1.1 Weigh 1.00 g of HAuCl₄ and dissolve it in 100 mL of methanol to obtain a 0.03M solution A;
2.1.2 Weigh 0.57 g of L-NIBC and dissolve it in 100 mL of glacial acetic acid (acetic acid) to obtain a 0.03M solution B;
2.1.3 Measure 1 mL of solution A, mix it with 0.5 mL, 1 mL, 2 mL, 3 mL, 4 mL, or 5 mL of solution B respectively (i.e. the mole ratio between HAuCl₄ and L-NIBC is 1:0.5, 1:1, 1:2, 1:3, 1:4, 1:5 respectively), react in an ice bath under stirring for 2 h, quickly add 1 mL of freshly prepared 0.03M (prepared by weighing 11.3 mg of NaBH 4 and dissolving it in 10 mL of ethanol) NaBH₄ ethanol solution when the solution turns colorless from bright yellow, continue the reaction for 30 min after the solution turns dark brown, and add 10 mL of acetone to terminate the reaction.
2.1.4 After the reaction, the reaction solution is subjected to gradient centrifugation to obtain L-NIBC-AuCs powder with different particle sizes. Specific method: After the reaction is completed, the reaction solution is transferred to an ultrafiltration tube with MWCO of 30K and a volume of 50 mL, and centrifuged at 10000 r/min for 20 min, and the retentate in the inner tube is dissolved in ultrapure water to obtain powder with a particle size of about 2.6 nm. Then, the mixed solution in the outer tube is transferred to an ultrafiltration tube with a volume of 50 mL and MWCO of 10K, and centrifuged at 13,000 r/min for 30 min. The retentate in the inner tube is dissolved in ultrapure water to obtain powder with a particle size of about 1.8 nm. Then the mixed solution in the outer tube is transferred to an ultrafiltration tube with a volume of 50 mL and MWCO of 3K, and centrifuged at 17,500 r/min for 40 min. The retentate in the inner tube is dissolved in ultrapure water to obtain powder with a particle size of about 1.1 nm.
2.1.5 Precipitate the powder in three different particle sizes obtained by gradient centrifugation, remove the solvent respectively, blow the crude product dry with N₂, dissolve it in 5 mL of ultrapure water, put it in a dialysis bag (MWCO is 3 KDa), put the dialysis bag in 2 L of ultrapure water, change water every other day, dialyze it for 7 days, freeze-dry it and keep it for future use.

2.2 Characterization of L-NIBC-AuCs

Characterization experiment was conducted for the powder obtained above (L-NIBC-AuCs). Meanwhile, ligand L-NIBC-modified gold nanoparticles (L-NIBC-AuNPs) are used as control. The method for preparing gold nanoparticles with ligand being L-NIBC refers to the reference (W. Yan, L. Xu, C. Xu, W. Ma, H. Kuang, L. Wang and N. A. Kotov, Journal of the American Chemical Society 2012, 134, 15114; X. Yuan, B. Zhang, Z. Luo, Q. Yao, D. T. Leong, N. Yan and J. Xie, Angewandte Chemie International Edition 2014, 53, 4623).

2.2.1 Observation of the Morphology by Transmission Electron Microscope (TEM)

The test powders (L-NIBC-AuCs sample and L-NIBC-AuNPs sample) were dissolved in ultrapure water to 2 mg/L as samples, and then test samples were prepared by hanging drop method. More specifically, 5 μL of the samples were dripped on an ultrathin carbon film, volatized naturally till the water drop disappeared, and then observe the morphology of the samples by JEM-2100F STEM/EDS field emission high-resolution TEM.

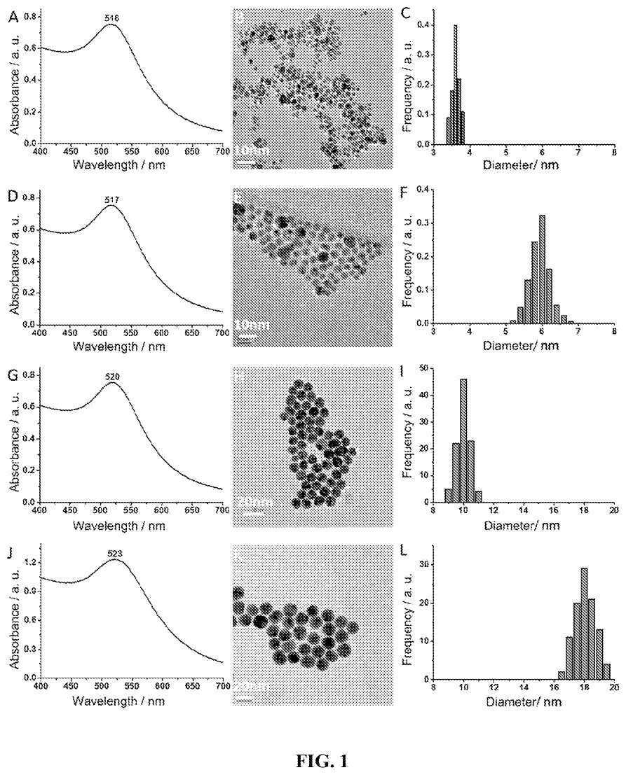

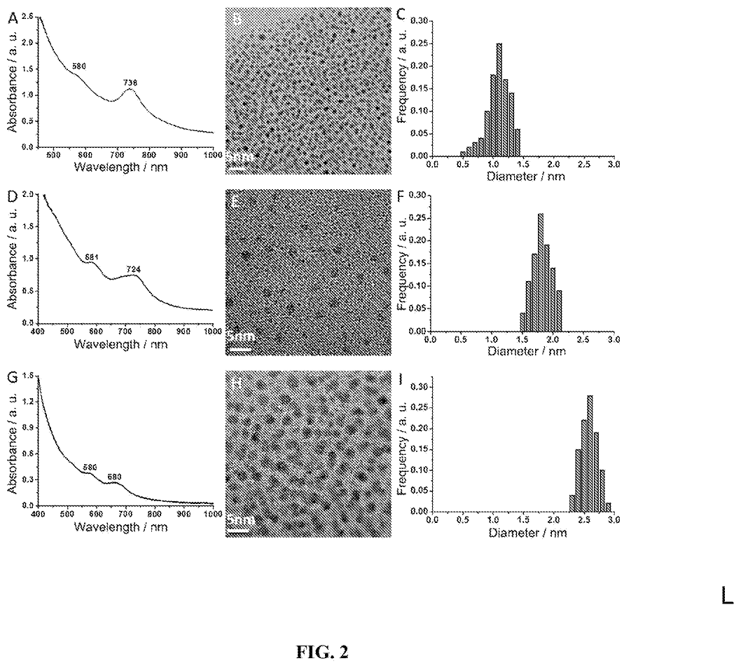

The four TEM images of L-NIBC-AuNPs are shown in panels B, E, H, and K of FIG. 1; the three TEM images of L-NIBC-AuCs are shown in panels B, E, and H of FIG. 2.

The images in FIG. 2 indicate that each of L-NIBC-AuCs samples has a uniform particle size and good dispersibility, and the average diameter of L-NIBC-AuCs (refer to the diameter of gold core) is 1.1 nm, 1.8 nm and 2.6 nm respectively, in good accordance with the results in panels C, F and I of FIG. 2. In comparison, L-NIBC-AuNPs samples have a larger particle size. Their average diameter (refer to the diameter of gold core) is 3.6 nm, 6.0 nm, 10.1 nm and 18.2 nm respectively, in good accordance with the results in panels C, F, I and L of FIG. 1.

2.2.2 Ultraviolet (UV)-Visible (Vis) Absorption Spectra

The test powders (L-NIBC-AuCs sample and L-NIBC-AuNPs sample) were dissolved in ultrapure water till the concentration was 10 mg·L⁻¹, and the UV-vis absorption spectra were measured at room temperature. The scanning range was 190-1100 nm, the sample cell was a standard quartz cuvette with an optical path of 1 cm, and the reference cell was filled with ultrapure water.

The UV-vis absorption spectra of the four L-NIBC-AuNPs samples with different sizes are shown in panels A, D, G and J of FIG. 1, and the statistical distribution of particle size is shown in panels C, F, I and L of FIG. 1; the UV-vis absorption spectra of three L-NIBC-AuCs samples with different sizes are shown in panels A, D and G of FIG. 2, and the statistical distribution of particle size is shown in panels C, F and I of FIG. 2.

FIG. 1 indicates that due to the surface plasmon effect, L-NIBC-AuNPs had an absorption peak at about 520 nm. The position of the absorption peak is relevant with particle size. When the particle size is 3.6 nm, the UV absorption peak appears at 516 nm; when the particle size is 6.0 nm, the UV absorption peak appears at 517 nm; when the particle size is 10.1 nm, the UV absorption peak appears at 520 nm, and when the particle size is 18.2 nm, the absorption peak appears at 523 nm. None of the four samples has any absorption peak above 560 nm.

FIG. 2 indicates that in the UV absorption spectra of three L-NIBC-AuCs samples with different particle sizes, the surface plasmon effect absorption peak at 520 nm disappeared, and two obvious absorption peaks appeared above 560 nm and the positions of the absorption peaks varied slightly with the particle sizes of AuCs. This is because AuCs exhibit molecule-like properties due to the collapse of the face-centered cubic structure, which leads to the discontinuity of the density of states of AuCs, the energy level splitting, the disappearance of plasmon resonance effect and the appearance of a new absorption peak in the long-wave direction. It could be concluded that the three powder samples in different particle sizes obtained above are all ligand-bound AuCs.

2.2.3 Fourier Transform Infrared Spectroscopy

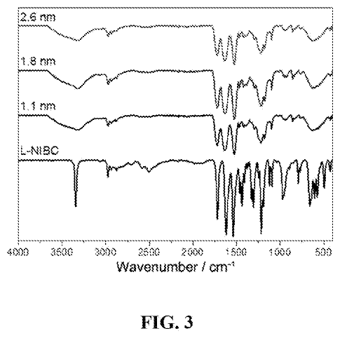

Infrared spectra were measured on a VERTEX80V Fourier transform infrared spectrometer manufactured by Bruker in a solid powder high vacuum total reflection mode. The scanning range is 4000-400 cm⁻¹ and the number of scans is 64. Taking L-NIBC-AuCs samples for example, the test samples were L-NIBC-AuCs dry powder with three different particle sizes and the control sample was pure L-NIBC powder. The results are shown in FIG. 3.

FIG. 3 shows the infrared spectrum of L-NIBC-AuCs with different particle sizes. Compared with pure L-NIBC (the curve at the bottom), the S—H stretching vibrations of L-NIBC-AuCs with different particle sizes all disappeared completely at 2500-2600 cm⁻¹, while other characteristic peaks of L-NIBC were still observed, proving that L-NIBC molecules were successfully bound to the surface of AuCs via Au—S bond. The figure also shows that the infrared spectrum of the ligand-bound AuCs is irrelevant with its size.

AuCs bound with other ligands were prepared by a method similar to the above method, except that the solvent of solution B, the feed ratio between HAuCl₄ and ligand, the reaction time and the amount of NaBH₄ added were slightly adjusted. For example: when L-cysteine, D-cysteine, N-isobutyryl-L-cysteine (L-NIBC) or N-isobutyryl-D-cysteine (D-NIBC) is used as the ligand, acetic acid is selected as the solvent; when dipeptide CR, dipeptide RC or 1-[(2S)-2-methyl-3-thiol-1-oxopropyl]-L-proline is used as the ligand, water is selected as the solvent, and so on and so forth; other steps are similar, so no further details are provided herein.

The present invention prepared and obtained a series of ligand-bound AuCs by the foregoing method. The ligands and the parameters of the preparation process are shown in Table 1.

TABLE 1
Preparation parameters of AuCs bound with different ligands in the present invention

| | | Parameter | | | | | | |
|---|---|---|---|---|---|---|---|---|
| | | | | Time of | | | Time of | |
| | | | | reaction in | | | reaction in | |
| | | | Feed ratio | an ice bath | | Mole ratio | an ice bath | |
| | | | between | under stirring | | between | under stirring | |
| | | Solvent used | HAuCl₄ and | before addition | | HAuCl₄ and | after addition | |
| | Ligand | for solution B | ligand | of NaBH₄ | | NaBH₄ | of NaBH₄ | |
| 1 | L-cysteine | Acetic acid | 1:3 | 2 | h | 1:2 | 0.5 | h |
| 2 | D-cysteine | Acetic acid | 1:3 | 2 | h | 1:2 | 0.5 | h |
| 3 | N-acetyl-L-cysteine | Ethanol | 1:4 | 1 | h | 1:1 | 0.5 | h |
| 4 | N-acetyl-D-cysteine | Ethanol | 1:4 | 1 | h | 1:1 | 0.5 | h |
| 5 | L-NIBC | Water | 1:4 | 0.5 | h | 1:2 | 0.5 | h |
| 6 | D-NIBC | Water | 1:4 | 0.5 | h | 1:2 | 0.5 | h |
| 7 | Other cysteine | Soluble solvent | 1:(0.1~100) | 0.5 h~24 h | | 1:(0.1~100) | 0.1~24 | h |
| | derivatives | | | | | | | |
| 8 | CR | Water | 1:4 | 22 | h | 2:1 | 0.5 | h |
| 9 | RC | Water | 1:4 | 20 | h | 2:1 | 0.5 | h |
| 10 | HC | Water | 1:3 | 12 | h | 1:2 | 2 | h |
| 11 | CH | Ethanol | 1:4 | 16 | h | 1:3 | 3 | h |
| 12 | GSH | Water | 1:2 | 12 | h | 1:1 | 3 | h |
| 13 | KCP | Water | 1:3 | 15 | h | 1:2 | 1 | h |
| 14 | PCR | Water | 1:4 | 16 | h | 1:3 | 2 | h |
| 15 | GSCR | Water | 1:4 | 16 | h | 1:3 | 1.5 | h |
| 16 | GCSR | Water | 1:3 | 12 | h | 1:2 | 2 | h |
| 17 | CDEVD | Water | 1:7 | 1 | h | 1:0.1 | 0.5 | h |
| 18 | DEVDC | Water | 1:7 | 1 | h | 1:0.1 | 0.5 | h |
| 19 | Other oligopeptides | Soluble solvent | 1:(0.1~100) | 0.5 h~24 h | | 1:(0.1~100) | 0.1~24 | h |
| | containing cysteine | | | | | | | |
| 20 | 1-[(2S)-2-methyl-3- | Water | 1:8 | 2 | h | 1:7 | 1 | h |
| | thiol-1-oxopropyl]-L- | | | | | | | |
| | proline | | | | | | | |
| 21 | Mercaptoethanol | Ethanol | 1:2 | 2 | h | 1:1 | 1 | h |
| 22 | Thioglycollic acid | Acetic acid | 1:2 | 2 | h | 1:1 | 1 | h |
| 23 | Thiophenol | Ethanol | 1:5 | 5 | h | 1:1 | 1 | h |
| 24 | D-3-trolovol | Water | 1:2 | 2 | h | 1:1 | 1 | h |
| 25 | N-(2- | Water | 1:2 | 2 | h | 1:1 | 1 | h |
| | mercaptopropionyl)- | | | | | | | |
| | glycine | | | | | | | |
| 26 | Dodecyl mercaptan | Methanol | 1:5 | 5 | h | 1:1 | 1 | h |
| 27 | 2-aminoethanethiol | Water | 1:5 | 2 | h | 8:1 | 0.5 | h |
| | (CSH) | | | | | | | |
| 28 | 3-mercaptopropionic | Water | 1:2 | 1 | h | 5:1 | 0.5 | h |
| | acid (MPA) | | | | | | | |
| 29 | 4-mercaptobenoic | Water | 1:6 | 0.5 | h | 3:1 | 2 | h |
| | acid (p-MBA) | | | | | | | |
| 30 | Other compounds | Soluble solvent | 1:(0.01~100) | 0.5 h~24 h | | 1:(0.1~100) | 0.1~24 | h |
| | containing thiol | | | | | | | |

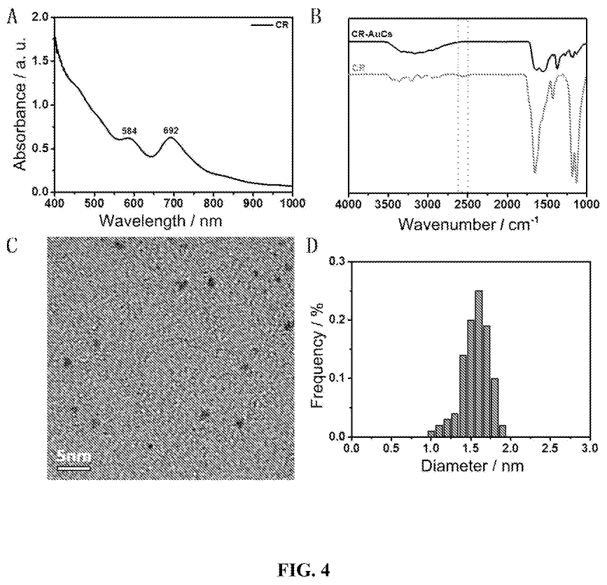

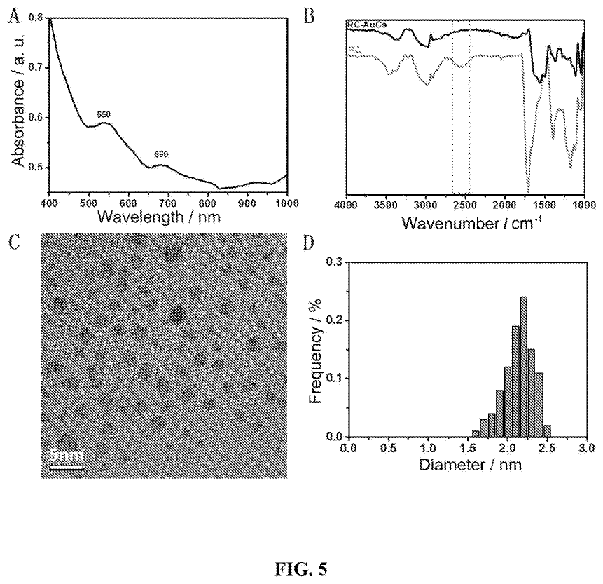

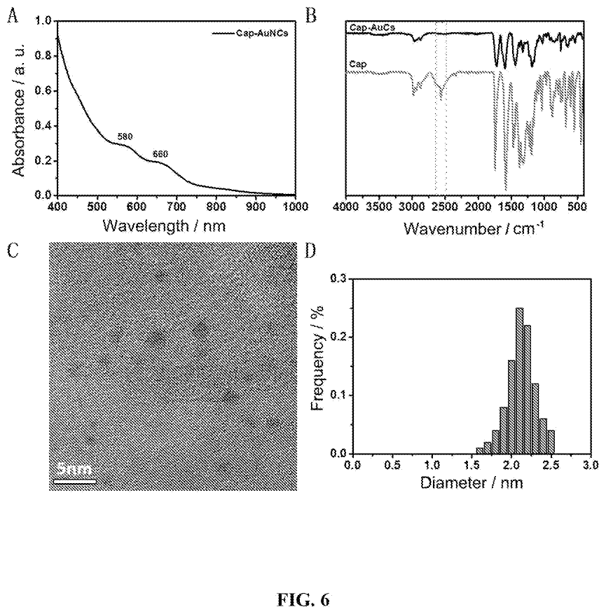

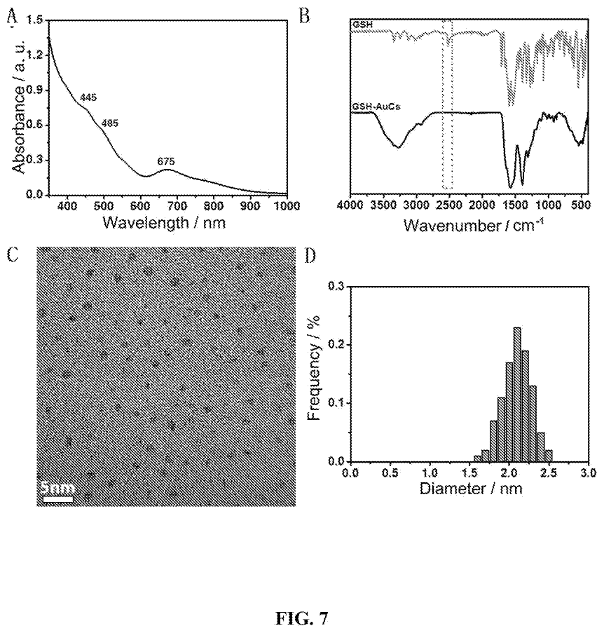

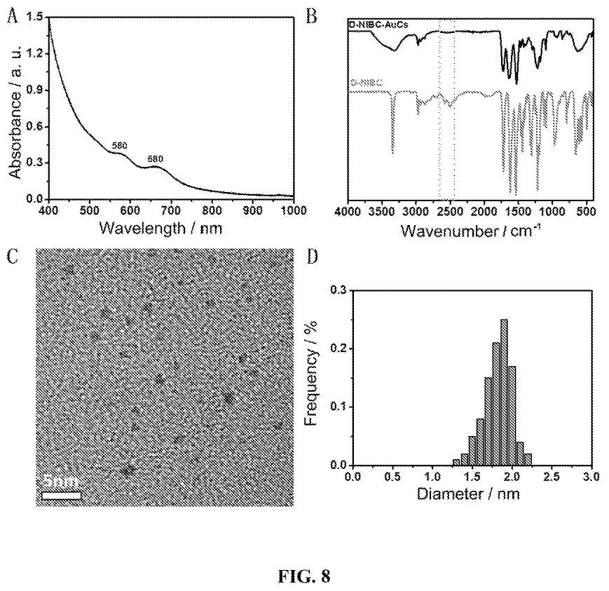

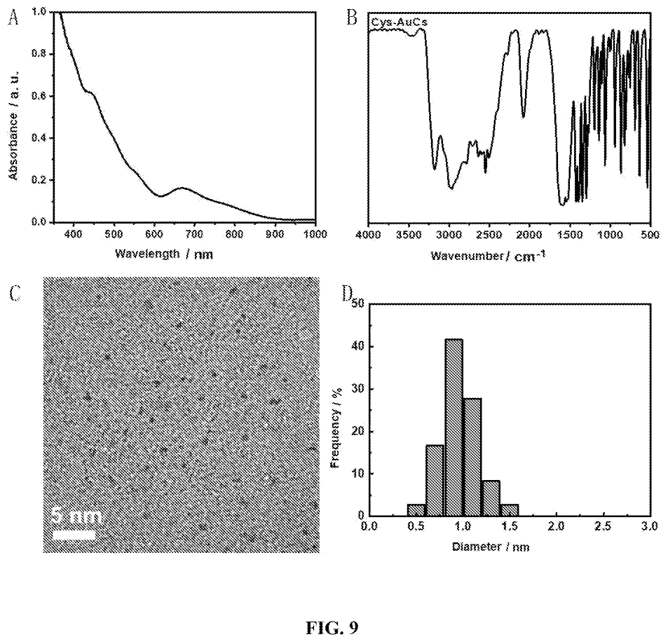

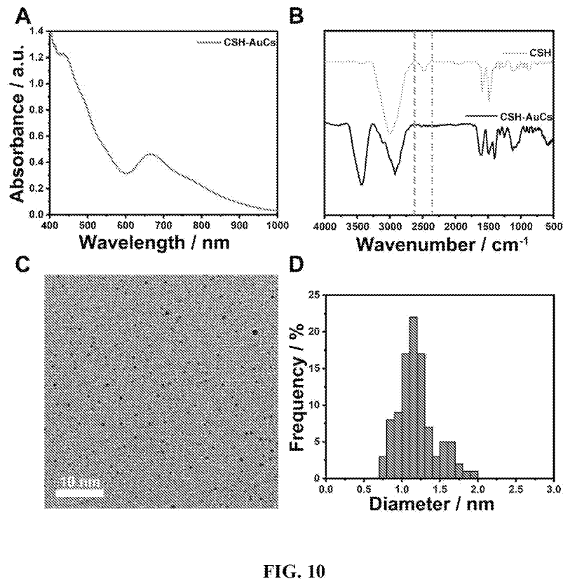

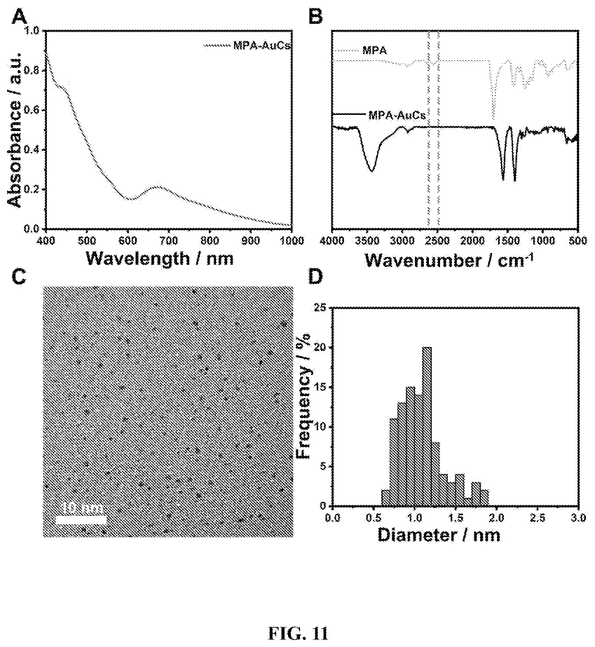

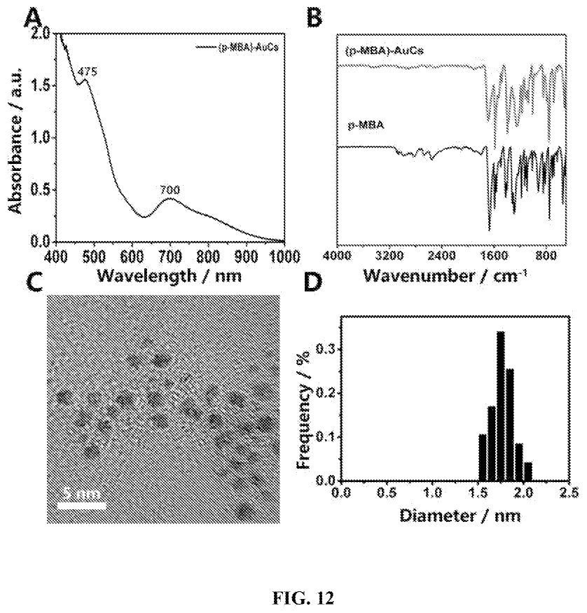

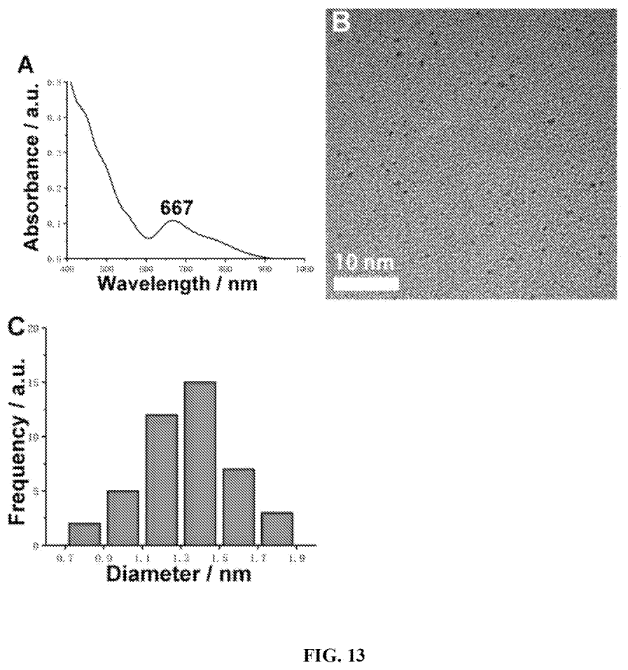

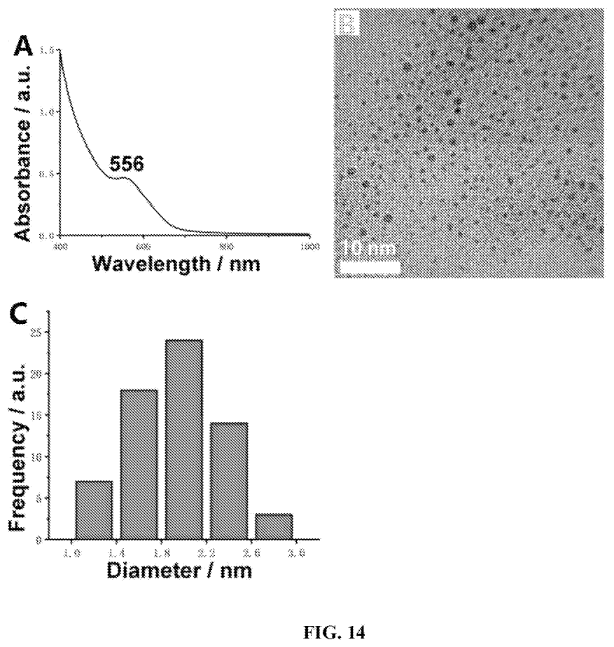

The samples listed in Table 1 are confirmed by the foregoing methods. The characteristics of eleven (11) different ligand-bound AuCs are shown in FIG. 4 (CR-AuCs), in FIG. 5 (RC-AuCs), in FIG. 6 (Cap-AuCs) (Cap denotes 1-[(2S)-2-methyl-3-thiol-1-oxopropyl]-L-proline), in FIG. 7 (GSH-AuCs), in FIG. 8 (D-NIBC-AuCs), in FIG. 9 (L-Cys-AuCs), in FIG. 10. (CSH-AuCs), in FIG. 11 (MPA-AuCs), in FIG. 12 (p-MBA-AuCs), in FIG. 13 (CDEVD-AuCs), and in FIG. 14 (DEVDC-AuCs). FIGS. 4-12 show UV spectra (panel A), infrared spectra (panel B), TEM images (panel C), and particle size distribution (panel D). FIGS. 13 and 14 show UV spectra (panel A), TEM images (panel B), and particle size distribution (panel C).

The results indicate that the diameters of AuCs bound with different ligands obtained from Table 1 are all smaller than 3 nm. Ultraviolet spectra also show disappearance of peak at 520±20 nm, and appearance of absorption peak in other positions. The position of the absorption peak could vary with ligands and particle sizes as well as structures. In certain situations, there is no special absorption peak, mainly due to the formation of AuCs mixtures with different particles sizes and structures or certain special AuCs that moves the position of absorption peak beyond the range of UV-vis spectrum. Meanwhile, Fourier transform infrared spectra also show the disappearance of ligand thiol infrared absorption peak (between the dotted lines in panel B of FIGS. 4-8), while other infrared characteristic peaks are all retained, suggesting that all ligand molecules have been successfully bound to gold atoms to form ligand-bound AuCs, and the present invention has successfully obtained AuCs bound with the ligands listed in Table 1.

### Embodiment 3. Testing Samples for Animal Studies

A1: ligand L-NIBC-bound gold clusters (L-NIBC-AuCs), gold core diameter in the range of 0.5-3.0 nm.
A2: ligand N-acetyl-L-cysteine-bound gold clusters (L-NAC-AuCs), gold core diameter in the range of 0.5-3.0 nm.
A3: ligand CR-bound gold clusters (CR-AuCs), gold core diameter in the range of 0.5-3.0 nm.
A4: ligand L-cysteine-bound gold clusters (L-C-AuCs), gold core diameter in the range of 0.5-3.0 nm.
A5: ligand DEVDC-bound gold clusters (DEVDC-AuCs), gold core diameter in the range of 0.5-3.0 nm.
B1: L-NIBC-bound gold nanoparticles (L-NIBC-AuNPs), size distribution range of 6.2±1.2 nm.

All testing samples were prepared following the above described method with slight modification, and their quality was characterized using the above described methods.

### Embodiment 4. Forced Swimming Test (FST)

In the FST, mice were placed in a cylindrical barrel with a water depth of 18 cm and a water temperature of 26° C. The mice swim in the barrel for 6 minutes. During this period, the floating time of mice is recorded by the instrument, and the floating time of the last 4 minutes is used for data analysis. The immobility (i.e. floating time) ratio (%) is calculated as 100× immobility time/test time. The higher the immobility ratio, the higher the depression degree of mice.

All data were analyzed by prism 6.01 software (one-way ANOVA plus Dunnett's multiple comparison test).

84 ICR mice were randomly divided into the following 7 groups (n-12): C, normal saline control group; A1 drug administration group (L-NIBC-AuCs); A2 drug administration group (L-NAC-AuCs); A3 drug administration group (CR-AuCs); A4 drug administration group (L-C-AuCs); A5 drug administration group (DEVDC-AuCs); and B1 drug administration group (L-NIBC-AuNPs). All drugs were dissolved in normal saline solution. The mice in A1, A2, A3, A4, A5 and B1 groups were intraperitoneally injected once a day at the dose of 20 mg/kg mice body weight and the injection volume was 50 μl, and the mice in the control group were intraperitoneally injected with the same volume of normal saline. On the 7th day (day 7), the drug was administered after the mice were being adapted to the laboratory environment for 60 minutes, and the Forced Swimming Test (FST) was conducted 30 minutes after administration.

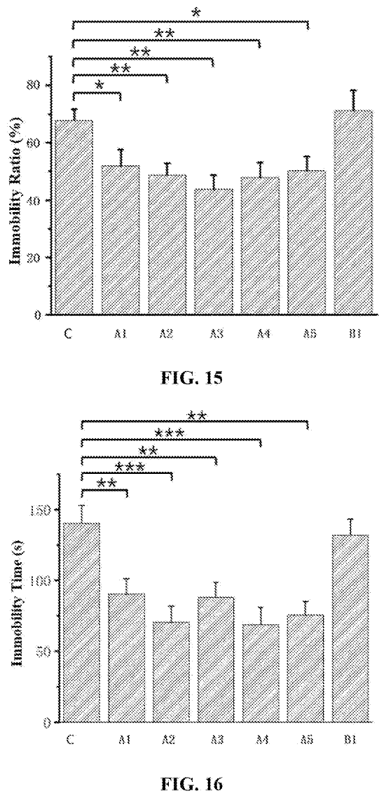

FIG. 15 shows the results of forced swimming test of mice in control group and each of drug administration groups. The control group of mice showed the immobility ratio of 67.8%±3.9%; the A1, A2, A3, A4 and A5 gold clusters administration groups of mice showed the immobility ratio of 52.0%±5.7%, 48.7%±4.0%, 43.9%±4.9%, 47.9%±5.2%, or 50.2%±5.0% respectively, significantly lower than that of the control group (*: P<0.05; **: P<0.01; ***: P<0.001). In contrast, B1 gold nanoparticle administration group of mice showed no significant change comparing to the control group.

### Embodiment 5. Tail Suspension Test

In the tail suspension test (TST) of mice, the tail end 2 cm of the mice is pasted on a horizontal wooden stick to make the animals hang. The distance of mice is 5 cm away from the surroundings. The immobility time (seconds) of mice in 6 minutes is observed and recorded. The longer the immobility time, the higher the depression degree of mice. All data are analyzed as described above.

84 ICR mice were used for tail suspension test. The grouping drug injection scheme were consistent with the forced swimming test as described above.

FIG. 16 shows the results of tail suspension test of mice in control group and each of drug administration groups. The control group of mice showed the immobility time of 140.3±12.4 seconds, while the A1, A2, A3, A4 and A5 gold clusters administration groups of mice showed the immobility time of 90.2±11.1 seconds, 70.4±11.4 seconds, 88.0±10.6 seconds, 68.8±12.2 seconds and 75.5±9.7 seconds, respectively, significantly decreased comparing with the control group ((*: P<0.05; ** : P<0.01; *** : P<0.001). In contrast, B1 gold nanoparticle administration group showed no significant difference than the control group.

The results show that the five gold cluster drugs have significant treatment effects on depression, but gold nanoparticles are ineffective.

### Embodiment 6. Social Stress Animal Model Test

6.1. Modeling and Drug Administration

The social stress animal model simulates a situation where humans encounter frustration with isolation and helplessness in normal communication, allowing aggressive CD-1 mice to attack C57BL/6J mice (abbreviated as C57) for a short period of time, and letting C57 mice be in the threat and fear of aggressive CD-1 mice for a long time. When mice are repeatedly exposed to the pressure of social failure, it will cause obvious depression-like manifestations characterized by lack of interest, anxiety and social avoidance behavior. This model is an animal model that is closer to the etiology of human depression.

First, C57 normal mice were challenged with aggressive CD-1 mice.
(1) the aggressive CD-1 mice were placed on one side of a mouse cage separated by a perforated transparent partition, and raised for 24 hours;
(2) the normal C57 mice according to their numbering were put into in the same side with the aggressive CD-1 mice in the mouse cage, and stimulated for 5 minutes (avoiding injury); then C57 mice were taken out and put into the other side of the same mouse cage separated by a perforated transparent partition (the two sides were separated by the transparent partition), so that C57 mice could see the aggressive CD-1 mice and smell the odor of CD-1 mice; the C57 mice and CD-1 mice stayed along overnight for 24 hours;
(3) during the experiment, each C57 mouse was continuously stressed for 20 days. Within 20 days, C57 mice of different model groups according to their numbering were placed in aggressive CD-1 mouse cages to receive stimulation every day. For 20 days, no more than one contact was made between any two mice (that is, no repeated contact to avoid familiarity);
(4) in the normal control group, C57 mice were used to replace aggressive CD-1 mice, separated by transparent plates, and C57 mice in the normal control group were replaced daily according to the numbering;
(5) at the end of the last day of modeling, the modeled C57 mice and the normal control group C57 mice were reared in single cages for 24 hours separately, and then behavioral tests were performed.

On the 15th day of social stress, the modeled C57 mice were administered with drugs A1, A2, A3, A4, A5 and B1 for 9 consecutive days, once a day by intraperitoneal injection at a dose of 20 mg/kg, giving rise to the designation of A1 administration group, A2 administration group, A3 administration group, A4 administration group, A5 administration group, and B1 administration group. Starting on the 7th day of administration (the 21st day from the modeling), behavioral tests were performed one hour after the drug administration. The normal control group and the model control group were given the same volume of physiological saline solution, and behavioral tests were performed on the corresponding days. Each group had 15 mice.

6.2. Behavioral Tests

The first behavioral test was the social behavior test, which was used to detect social avoidance behavior (a typical feature of depression) in mice. This test was conducted respectively at 14^{th} day and 21^{st} day from the beginning of modeling. The first test at 14^{th} day was to evaluate whether the modeling was successful, and the second test at 21^{st} day to assay the effects of drugs on the social behavior of mice.

The social behavior test consisted of two stages, each stage 2.5 min with an interval of 30 s. In the first stage (no target stage), a gas-permeable cylinder with a radius of 4 cm is placed on the side of the open field, and the area within 8 cm from the center of the cylinder is defined as the interaction zone (IZ). The time spent in the interaction zone of the successfully modeled C57 mice was recorded and designated as T1. In the second stage (the target stage), a CD-1 mouse that has not been in contact with the mouse during the modeling stage is placed in the cylinder, allowing visual and olfactory interaction between the two (but not allowing physical contact), the visual and olfactory interaction time between the two at this stage in the interaction zone was recorded, and designated it as T2. The ratio of T2 to T1 (T2/T1) is called the social interaction rate (SIR). The smaller the value, the more obvious the social avoidance behavior. Conversely, the better the antidepressant ability of the drug is.

The second behavioral test was the elevated cross maze test, which was used to detect anxiety (another typical feature of depression) behavior in mice. The elevated cross maze test started at 23^{rd} day of modeling. Modeled C57 mice were placed on the platform of the elevated cross maze; then, the movement time, movement distance and shuttling times of the mice in each arm within 5 minutes were observed and recorded. The following data were calculated: (1) the percentage of the time spent by mice in the open arm over the time spent by the mice in both the open arm and the closed arm: TO %=100%*open arm time/(open arm time+closed arm time); (2) the percentage of the movement distance in the open arm over the total movement distance: DO %=100%*open arm movement distance/(open arm movement distance+closed arm movement distance); (3) The percentage of the number of shuttling in the open arm to the total number of shuttling: EO %=100%*Open arm shuttling times/(open arm shuttling times+closed arm shuttling times). The larger the above values, the better the anti-anxiety ability of the drug is.

All data were analyzed by prism 6.01 software (one-way ANOVA plus Dunnett's multiple comparison test).

6.3. Test Results

6.3.1. Successful Establishment of the Model

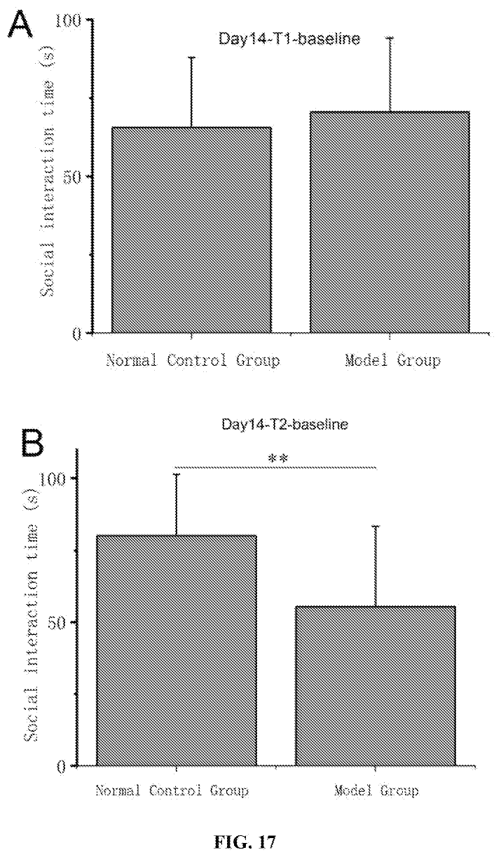

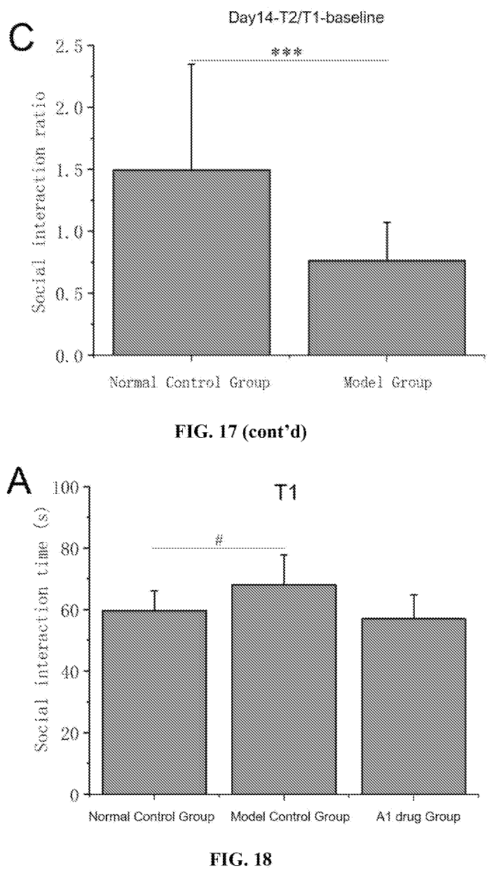

The social behavior ability of the mice was tested on the 14th day (1 day prior to drug administration). The results are shown in FIG. 17. FIG. 17A-FIG. 17C show the results of T1, T2 and T2/T1 of mice in the social behavior test. The results showed that the T2/T1 value of model mice was significantly lower than that of normal mice, and the difference was extremely significant (P<0.001, ** *), indicating that the social ability of model mice was significantly lower than that of normal mice, and the model was successfully established.

6.3.2. Effect of Drugs on Social Behavior of Model Mice

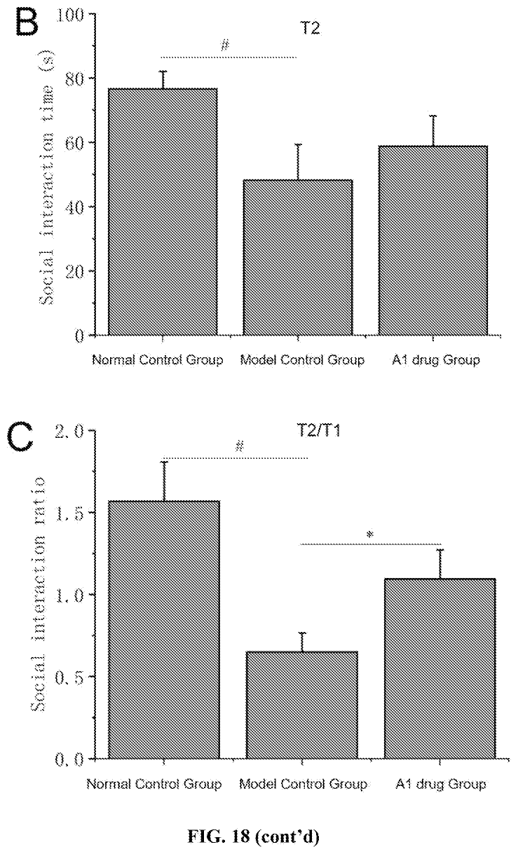

FIG. 18 shows the effect of gold cluster drugs on the social behavior test of mice with chronic social stress, taking A1 drug as an example. The results showed that A1-A5 five gold cluster drugs could significantly improve the T2/T1 ratio of model mice, and the difference was significant compared with the model control group (P<0.05, *), indicating that the five gold cluster drugs could effectively improve the social behavioral capabilities, and had significant antidepressant effect. However, compared with the model control group, the T2/T1 value of B1 group did not improve, indicating that it did not have antidepressant effect.

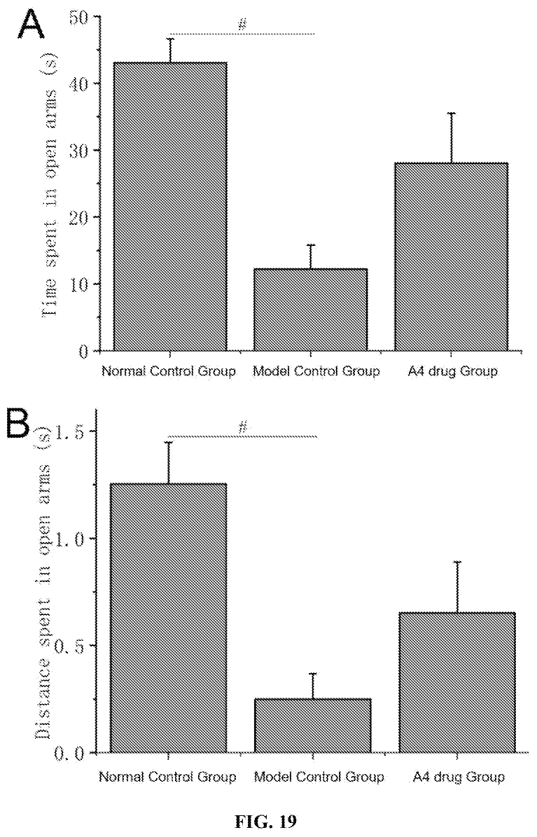

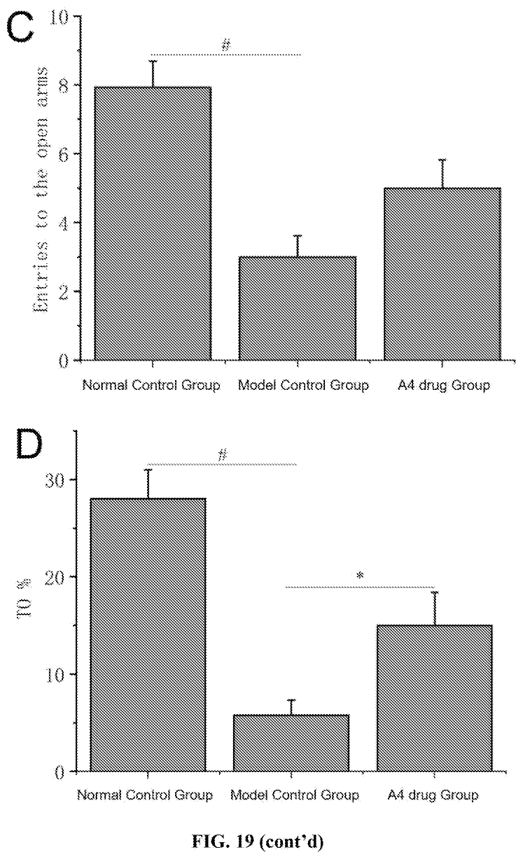

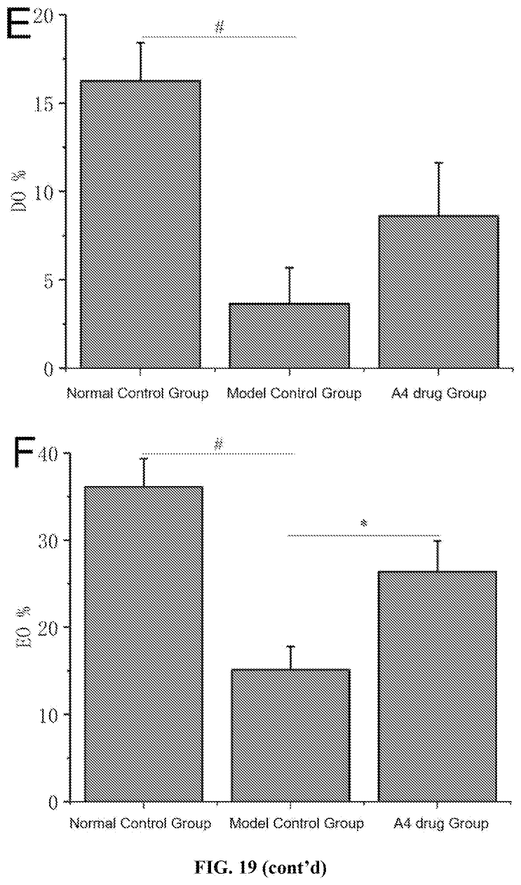

FIG. 19 shows the results of the effects of gold cluster drugs on mice with chronic social stress in the elevated cross maze test, taking A4 drug as an example. The results showed that, at 23^{rd} day of modeling, A1-A5 five gold clusters could significantly increase the movement time in the open arm (FIG. 19A), the movement distance in the open arm (FIG. 19B), the shuttling times in the open arm (FIG. 19C), the percentage (TO %) of the time in the open arm to the time in the open arm+closed arm (FIG. 19D), the percentage (DO %) of movement distance in the open arm to the total movement distance (FIG. 19E), and the percentage (EO %) of shuttling times in the open arm to the total shuttling times (FIG. 19F). Taking A4 drug as an example, the TO % and EO % values of the drug administration group had significant differences than the model control group (P<0.05, *). These results indicate that gold cluster drugs have obvious anti-anxiety ability. However, B1 drug had no improvement of these test data, indicating that gold nanoparticles larger than 3.0 nm had no effect on anxiety-like behavior of model mice.

These results indicate that gold cluster drugs can significantly improve the social behavior and anxiety-like behavior of model mice, and can be developed as antidepressant drugs. However, gold nanoparticles larger than 3.0 nm have no such effect, and cannot be used in the development of antidepressant drugs.

Other ligand-bound AuCs with different sizes also have the similar effects, while their effects vary to certain extents. They would not be described in detail here.

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the scope of the present invention. Accordingly, the scope of the present invention is defined by the appended claims and is supported by the foregoing description.

### REFERENCES

Da̧browska-Bouta B, Ziȩba M, Orzelska-Górka J, Skalska J, Sulkowski G, Frontczak-Baniewicz M, Talarek S, Listos J, Strużyńska L. Influence of a Low Dose of Silver Nanoparticles on Cerebral Myelin and Behavior of Adult Rats. Toxicology. 2016 Jul. 1; 363-364:29-36.
Saeidienik F, Shahraki M R, Fanaei H, Badini F. The Effects of Iron Oxide Nanoparticles Administration on Depression Symptoms Induced by LPS in Male Wistar Rats. Basic Clin Neurosci. 2018; 9(3):209-216.
Xie Y, Wang Y, Zhang T, Ren G, Yang Z. Effects of Nanoparticle Zinc Oxide on Spatial Cognition and Synaptic Plasticity in Mice With Depressive-Like Behaviors. J Biomed Sci. 2012 Feb. 3; 19(1):14.
Zhang X, Xu Y, Zhou L, Zhang C, Meng Q, Wu S, Wang S, Ding Z, Chen X, Li X, Chen R. Sex-Dependent Depression-Like Behavior Induced by Respiratory Administration of Aluminum Oxide Nanoparticles. Int J Environ Res Public Health. 2015 Dec. 9; 12(12):15692-705.

1. A method for treating depression in a subject, wherein the method comprises:
administering a composition to the subject with depression;
wherein the composition comprises a ligand-bound gold cluster; and
a pharmaceutically acceptable excipient;
wherein the ligand-bound gold cluster comprises:
a gold core; and
a ligand bound to the gold core.

2. The method of claim 1, wherein the gold core has a diameter in the range of 0.5-3 nm.

3. The method of claim 1, wherein, the gold core has a diameter in the range of 0.5-2.6 nm.

4. The method of claim 1, wherein the ligand is one selected from the group consisting of L-cysteine and its derivatives, D-cysteine and its derivatives, cysteine-containing oligopeptides and their derivatives, and other thiol-containing compounds.

5. The method of claim 4, wherein the L-cysteine and its derivatives are selected from the group consisting of L-cysteine, N-isobutyryl-L-cysteine (L-NIBC), and N-acetyl-L-cysteine (L-NAC), and wherein the D-cysteine and its derivatives are selected from the group consisting of D-cysteine, N-isobutyryl-D-cysteine (D-NIBC), and N-acetyl-D-cysteine (D-NAC).

6. The method of claim 4, wherein the cysteine-containing oligopeptides and their derivatives are cysteine-containing dipeptides, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

7. The method of claim 4, wherein the cysteine-containing oligopeptides and their derivatives are cysteine-containing tripeptides, wherein the cysteine-containing tripeptides are selected from the group consisting of glycine-L(D)-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

8. The method of claim 4, wherein the cysteine-containing oligopeptides and their derivatives are cysteine-containing tetrapeptides, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

9. The method of claim 4, wherein the cysteine-containing oligopeptides and their derivatives are cysteine-containing pentapeptide, wherein the cysteine-containing pentapeptides are selected from the group consisting of Cysteine-Aspartic acid-Glutamic acid-Valine-Aspartic acid (CDEVD) and Aspartic acid-Glutamic acid-Valine-Aspartic acid-Cysteine (DEVDC).

10. The method of claim 4, wherein the other thiol-containing compounds are selected from the group consisting of 1-[(2S)-2-methyl-3-thiol-1-oxopropyl]-L(D)-proline, thioglycollic acid, mercaptoethanol, thiophenol, D-3-trolovol, N-(2-mercaptopropionyl)-glycine, dodecyl mercaptan, 2-aminoethanethiol (CSH), 3-mercaptopropionic acid (MPA), and 4-mercaptobenzoic acid (p-MBA).

11. A pharmaceutical composition for treatment of depression in a subject, wherein the pharmaceutical composition comprises a ligand-bound gold cluster; and a pharmaceutically acceptable excipient;
wherein the ligand-bound gold cluster comprises:
a gold core; and
a ligand bound to the gold core.

12. The pharmaceutical composition of claim 11, wherein the gold core has a diameter in the range of 0.5-3 nm.

13. The pharmaceutical composition of claim 11, wherein the gold core has a diameter in the range of 0.5-2.6 nm.

14. The pharmaceutical composition of claim 11, wherein the ligand is one selected from the group consisting of L-cysteine and its derivatives, D-cysteine and its derivatives, cysteine-containing oligopeptides and their derivatives, and other thiol-containing compounds.

15. The pharmaceutical composition of claim 14, wherein the L-cysteine and its derivatives are selected from the group consisting of L-cysteine, N-isobutyryl-L-cysteine (L-NIBC), and N-acetyl-L-cysteine (L-NAC), and wherein the D-cysteine and its derivatives are selected from the group consisting of D-cysteine, N-isobutyryl-D-cysteine (D-NIBC), and N-acetyl-D-cysteine (D-NAC).

16. The pharmaceutical composition of claim 14, wherein the cysteine-containing oligopeptides and their derivatives are cysteine-containing dipeptides, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

17. The pharmaceutical composition of claim 14, wherein the cysteine-containing oligopeptides and their derivatives are cysteine-containing tripeptides, wherein the cysteine-containing tripeptides are selected from the group consisting of glycine-(D)L-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

18. The pharmaceutical composition of claim 14, wherein the cysteine-containing oligopeptides and their derivatives are cysteine-containing tetrapeptides, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

19. The pharmaceutical composition of claim 14, wherein the cysteine-containing oligopeptides and their derivatives are cysteine-containing pentapeptide, wherein the cysteine-containing pentapeptides are selected from the group consisting of Cysteine-Aspartic acid-Glutamic acid-Valine-Aspartic acid (CDEVD) and Aspartic acid-Glutamic acid-Valine-Aspartic acid-Cysteine (DEVDC).

20. The pharmaceutical composition of claim 14, wherein the other thiol-containing compounds are selected from the group consisting of 1-[(2S)-2-methyl-3-thiol-1-oxopropyl]-L(D)-proline, thioglycolic acid, mercaptoethanol, thiophenol, D-3-trolovol, N-(2-mercaptopropionyl)-glycine, dodecyl mercaptan, 2-aminoethanethiol (CSH), 3-mercaptopropionic acid (MPA), and 4-mercaptobenzoic acid (p-MBA).
